(19) United States
(12) Patent Application Publication
(10) Pub. No.: US 2026/0232561 A1
(43) Pub. Date: Aug. 13, 2026

(54) PEPTIDE ACTIVATING RETINOIC ACID RECEPTOR, AND COSMETIC COMPOSITION CONTAINING SAME

(71) Applicant: SUPADELIXIR INC., Chuncheon-si (KR)

(72) Inventors: Jang Hee HAHN, Chuncheon-si (KR); Min Seo KIM, Chuncheon-si (KR)

(21) Appl. No.: 18/834,611

(22) PCT Filed: Sep. 12, 2023
(86) PCT No.: PCT/KR2023/013670

§ 371 Date: Apr. 28, 2026

(30) Foreign Application Priority Data
Sep. 12, 2023 (KR) 10-2023-0121129

(51) Int. Cl. A61K 8/64; A61Q 19/02; A61Q 19/08; C07K 5/097

(52) U.S. Cl. CPC A61K 8/64; A61Q 19/02; A61Q 19/08; C07K 5/0823

(57) ABSTRACT

Proposed are a peptide activating retinoic acid receptors and a cosmetic composition containing the same.

### TECHNICAL FIELD

The present disclosure relates to a peptide activating retinoic acid receptors and a cosmetic composition containing the same.

### BACKGROUND ART

Retinoic acid receptors are receptors required for the action of retinoic acid, which may be considered the active form of vitamin A. The retinoic acid receptors bind to retinoic acid and then to a specific location in a gene to control the transcription of that gene. In addition, the retinoic acid receptors are known to be involved in melanin production, wrinkle production, and anti-aging (skin rejuvenation) in the skin.

Accordingly, substances for treating related diseases by controlling the action of the retinoic acid receptors are being developed, as shown in the patent document below.

### Patent Document

Korean Patent Application Publication No. 10-1990-0700601 (published on Aug. 16, 1990), titled “Retinoic Acid Receptor Composition and Method for Producing Same”

However, substances that control the action of conventional retinoic acid receptors have limitations in that the substances are not effective enough or are mostly chemically synthesized and may cause side effects when used.

### DISCLOSURE

### Technical Problem

The present disclosure was devised to solve the problems.

The objective of the present disclosure is to provide a peptide activating retinoic acid receptors and a cosmetic composition using the same, which can be used to reduce wrinkles, whiten skin, and rejuvenate skin (anti-aging).

### Technical Solution

To achieve the described objective, the present disclosure is implemented by embodiments having the following configuration.

According to one embodiment of the present disclosure, a peptide according to the present disclosure is made of any one amino acid sequence represented by SEQ ID NO: 1 or 2.

According to another embodiment of the present disclosure, the peptide according to the present disclosure activates retinoic acid receptors.

According to yet another embodiment of the present disclosure, the peptide according to the present disclosure is used to whiten skin, reduce wrinkles, or rejuvenate skin.

According to yet another embodiment of the present disclosure, a cosmetic composition according to the present disclosure contains a peptide including any one amino acid sequence represented by SEQ ID NO: 1 or 2.

According to one embodiment of the present disclosure, a peptide according to the present disclosure includes any one amino acid sequence represented by SEQ ID NO: 1 or 2.

According to yet another embodiment of the present disclosure, the cosmetic composition to whiten skin according to the present disclosure contains a peptide including an amino acid sequence.

According to yet another embodiment of the present disclosure, a cosmetic composition to reduce skin wrinkles according to the present disclosure contains a peptide including any one amino acid sequence represented by SEQ ID NO: 1 or 2.

### Advantageous Effects

The following effects can be achieved by the described embodiments of the present disclosure.

The present disclosure activates the retinoic acid receptors using a peptide including an amino acid sequence represented by SEQ ID NO: 1 or 2, thereby exhibiting the effects of reducing wrinkles, whitening skin, and rejuvenating (anti-aging) skin.

### DESCRIPTION OF DRAWINGS

FIG. 1 is a graph showing the effects of peptides on the physical binding between RAR and RXR according to one embodiment of the present disclosure;

FIG. 2 is a graph showing the effects of peptides on the physical binding between P53 and POLII according to another embodiment of the present disclosure; and

FIG. 3 is a graph showing the effects of peptides on the induction of intracellular collagen synthesis according to yet another embodiment of the present disclosure.

### BEST MODE

Hereinafter, peptides activating retinoic acid receptors according to the present disclosure and cosmetic compositions containing the same will be described in detail with reference to the attached drawings. Unless otherwise specified, all terms in this specification have the same general meaning as understood by those skilled in the art to which the present disclosure pertains, and when there is a conflict with the meaning of the terms used herein, this specification follows the definitions used in the specification. Additionally, detailed descriptions of well-known functions and configurations that may unnecessarily obscure the gist of the present disclosure are omitted. Throughout the specification, when a part “includes” a certain component, this means that the part may further include other components rather than excluding other components unless specifically stated to the contrary.

According to one embodiment, the present disclosure relates to peptides activating retinoic acid receptors. The peptides are made of the amino acid sequence of SEQ ID NO: 1 (Ile-Gln-Glu-Met) or SEQ ID NO: 2 (Thr-Val-Glu-Phe). The peptides may improve skin conditions by activating retinoic acid receptors. Specifically, the peptides may promote skin wrinkle reduction, skin whitening, and skin rejuvenation (anti-aging). The peptides may be used for cosmetic compositions and therapeutic compositions.

According to another embodiment, the present disclosure relates to cosmetic compositions containing a peptide including any one amino acid sequence represented by SEQ ID NO: 1 or 2. The cosmetic compositions may reduce skin wrinkles, whiten skin, and promote skin rejuvenation (anti-aging). Various known ingredients that may provide formulation stability and additional skin condition improvement effects may be further included in the cosmetic compositions.

Hereinafter, the present disclosure will be described in more detail through examples. However, these are only for explaining the present disclosure in more detail, and the scope of the present disclosure is not limited thereto.

### <Example 1> Synthesis of Peptides

Peptides represented by SEQ ID NOs: 1 to 2 listed in Table 1 below were synthesized by an FMOC solid-phase method using an automated synthesizer (PeptrEx-R48, Peptron, Korea). The synthesized peptides were purified and analyzed by reverse-phase high-performance liquid chromatography (Prominence LC-20AB, Shimadzu, Japan) using a C18 analytical RP column (Shiseido Capcell Pak). The synthesized peptides were identified by mass spectrometry (HP 1100 Series LC/MSD, Hewlett-Packard, USA).

TABLE 1

| Peptide name | Sequence number | Amino acid sequence |
|---|---|---|
| Raragin A | Sequence number 1 | Ile-Gln-Glu-Met |
| Raragin B | Sequence number 2 | Thr-Val-Glu-Phe |

### <Example 2> Confirmation of Whether Peptides of Example 1 Activate Retinoic Acid Receptors to Increase Physical Binding Between RAR and RXR

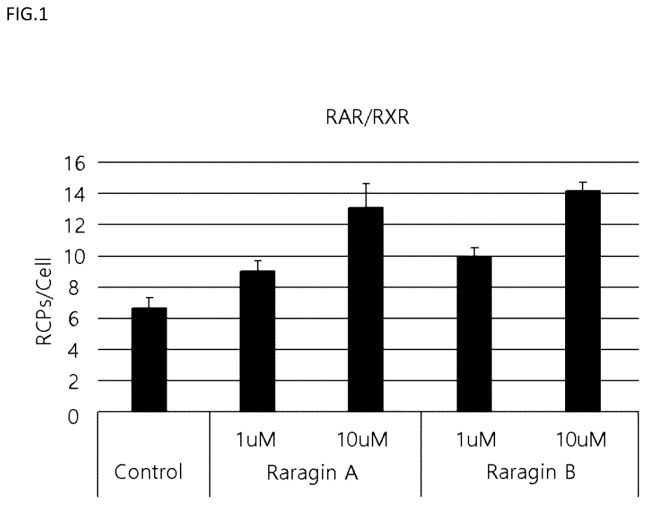

1. Retinol regulates the transcription of target DNA by inducing the binding between Retinoic acid receptor (RAR) and Retinoid X Receptor (RXR). To confirm whether the peptides of Example 1 activate the binding between RXR/RAR, the effects of the peptides on the physical binding between RAR and RXR were evaluated using an in-situ proximity ligation assay (PLA) method.
2. Specifically, 4.5×10⁴ HaCaT cells (human keratinocytes) were seeded into each well of a 24-well microplate with DMEM serum medium. The cells were cultured in an incubator at a temperature of 37° C. and with 5% CO₂ for 24 hours. Next, the cells were treated with each of the peptides of Example 1, and the cells were incubated under the same conditions for 30 minutes. The concentration of each peptide in the medium was set to 1 and 10 μM. The control group received no treatment. Afterward, the cells in each well were washed with PBS and fixed by treatment with 2% formaldehyde for 15 minutes, and then the cells were treated with 0.1% TritonX-100 for 5 minutes to increase antibody permeability into the cells. Anti-RAR monoclonal antibodies (Santa Cruz, USA) and anti-RXR polyclonal antibodies (Santa Cruz, USA) were added, and PLA probes were applied using an in-situ PLA kit (Sigma-Aldrich). Subsequently, hybridization, ligation, amplification, and mounting steps were performed.
3. Afterward, the physical interaction of RAR and RXR antibodies was quantified by measuring luminescence signals (PLA signals) detected in each cell using a fluorescence microscope (Logosbio/CelenaS, Korea), and the results are shown in FIG. 1.
4. As seen in FIG. 1, when the cells were treated with Raragin A to B of Example 1, the physical binding between RAR and RXR increased compared to the untreated control group. That is, Raragin A to B activated RAR and increased the physical binding between RAR and RXR, resulting in the same efficacy as retinol.

### <Example 3> Confirmation of Whether Peptides of Example 1 Activate Retinoic Acid Receptors to Increase Physical Binding Between P53 and POLII

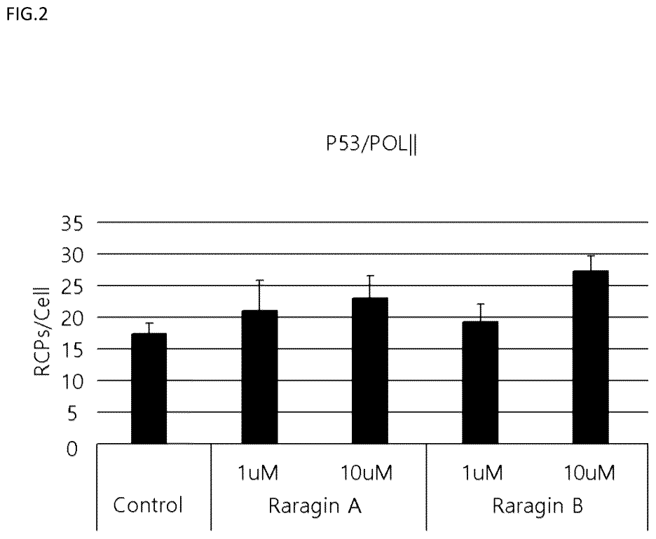

1. To confirm whether the peptides of Example 1 increase the transcriptional activity of P53, which was related to cell anti-aging, cells were treated with the peptides of Example 1, and then the effects of the peptides on the physical binding between P53 and POLII (RNA Polymerase II) was evaluated using an in-situ proximity ligation assay (PLA) method.
2. Specifically, 4.5×10⁴ HaCaT cells (human keratinocytes) were seeded into each well of a 24-well microplate with DMEM serum medium. The cells were cultured in an incubator at a temperature of 37° C. and with 5% CO₂ for 24 hours. Next, the cells were treated with each of the peptides of Example 1, and the cells were incubated under the same conditions for 30 minutes. The concentration of each peptide in the medium was set to 1 and 10 μM. The control group received no treatment. Afterward, the cells in each well were washed with PBS and fixed by treatment with 2% formaldehyde for 15 minutes, and then the cells were treated with 0.1% TritonX-100 for 5 minutes to increase antibody permeability into the cells. Anti-P53 polyclonal antibodies (CST, USA) and anti-POLII monoclonal antibodies (Santa Cruz, USA) were added, and PLA probes were applied using an in-situ PLA kit (Sigma-Aldrich). Subsequently, hybridization, ligation, amplification, and mounting steps were performed.
3. Afterward, the physical interaction of P53 and POLII antibodies was quantified by measuring luminescence signals (PLA signals) detected in each cell using a fluorescence microscope (Logosbio/CelenaS, Korea), and the results are shown in FIG. 2.
4. As seen in FIG. 2, when the cells were treated with Raragin A to B of Example 1, the physical binding between P53 and POLII increased compared to the untreated control group. That is, Raragin A and B prevented cell aging by increasing the transcriptional activity of P53.

### <Example 4> Evaluation of Whether Peptides of Example 1 have Effects on Intracellular Collagen Synthesis

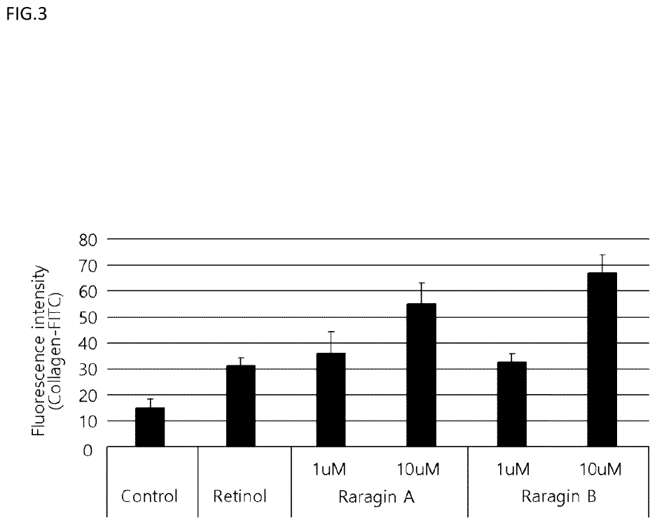

1. Immunofluorescence analysis was performed to evaluate the effects of the peptides of Example 1 on intracellular collagen synthesis.
2. Specifically, 4.5×10⁴ human dermal fibroblasts were seeded into each well of a 24-well microplate along with CEFOgro Human MSC Growth Medium). The cells were cultured in an incubator at a temperature of 37° C. and with 5% CO₂ for 24 hours. Next, the cells were treated with each of the peptides of Example 1, and the cells were incubated under the same conditions for 24 hours. The concentration of each peptide in the medium was set to 1 and 10 μM. Out of two control groups, one received no treatment, and the other was treated with 1 μM retinol. The cells in each well were washed with PBS and fixed by treatment with 4% formaldehyde for 15 minutes, and then the cells were treated with 0.1% TritonX-100 for 5 minutes to increase antibody permeability into the cells. The cells were washed with PBS solution and treated with anti-COL1A2 polyclonal antibodies (Santa Cruz, CA, USA) for 1 hour. Next, the cells were treated with mouse anti-goat IgG-FITC for 40 minutes. To stain the cytoskeleton, the cells were treated with Phalloidin-Rhodamin for 40 minutes. After mounting with DAPI-added reagent for cell nucleus staining, the cells were observed using a fluorescence microscope (Logosbio/CelenaS, Korea) and quantified using a program (NIS-Eliments-3.1). The results are shown in FIG. 3.
3. As seen in FIG. 3, when the cells were treated with Raragin A to B of Example 1, the collagen synthesis was significantly increased compared to the untreated control group.

### <Example 5> Evaluation of Whether Peptides of Example 1 Inhibit Melanin Synthesis in Cells

1. To evaluate the effects of the peptides of Example 1 on melanin synthesis in cells, it was evaluated whether the peptides of Example 1 inhibit melanin synthesis under the condition that melanin synthesis was promoted by stimulating melanin cells with α-MSH.
2. Specifically, 1×10⁵ B16F10 cells (mouse melanocytes) were seeded into each well of a 6-well microplate with DMEM serum medium. The cells were cultured in an incubator at a temperature of 37° C. and with 5% CO₂ for 24 hours. Next, the cells were treated with 100 nM of α-Melanocyte stimulating hormone (α-MSH) and the peptides of Example 1, respectively, and the cells were incubated under the same conditions for 72 hours. The concentration of each peptide in the medium was set to 1 and 10 μM. Out of three control groups, one received no treatment, another was treated with α-MSH alone, and the other was treated with 1 μM retinol. Afterward, to measure the amount of extracellular melanin, the supernatant was taken, and the absorbance of free melanin in the medium was measured at a wavelength of 400 nm. To measure the amount of intracellular melanin, the cells were washed with PBS after removing the medium, and then the cells were lysed with 1 M NaOH at a temperature of 60° C. for 30 minutes, and the absorbance was measured at a wavelength of 400 nm. The amount of free melanin and intracellular melanin measured by absorbance were quantified, and the results are shown in Table 2.
3. As seen in Table 2, when the cells were treated with Raragin A to B of Example 1, the increase in melanin induced by α-MSH stimulation was reduced compared to the untreated control group. That is, the peptides of Example 1 had a whitening effect.

TABLE 2

| | | | α-MSH + | α-MSH + Raragin | | | |
|---|---|---|---|---|---|---|---|
| | No | | Retinol | A | A | B | B |
| | treatment | α-MSH | (1 uM) | (1 uM) | (10 uM) | (1 uM) | (10 uM) |
| Melanin amount | 100 | 118 | 114 | 111 | 107 | 109 | 106 |
| (of Control) | | | | | | | |

### <Example 6> Skin Patch Experiment of Cosmetic Compositions Containing Peptides of Example 1

1. Cosmetic compositions were prepared by adding each of the peptides prepared in Example 1 to a commercially available solubilized ampoule base at a concentration of 50 ppm. Cosmetic composition 1 was a soluble ampoule base without the addition of peptides of Example 1. Cosmetic composition 2 was one with Raragin A. Cosmetic composition 3 was one with Raragin B.
2. Nine adults in their 20s to 60s were asked to apply the same amount of each of the cosmetic compositions to their faces every morning and evening for 6 weeks. After 6 weeks, a sensory evaluation of skin improvement was conducted, and the results are shown in Table 3. Sensory evaluation items included whether there was skin irritation, the degree of wrinkle reduction, the degree of skin tone improvement (whitening), and the degree of skin rejuvenation (comprehensively considering factors such as increased elasticity and scar reduction). The participants were asked to rate the condition on a scale from 0 to 5, with 5 indicating excellent condition and 0 indicating poor condition.
3. As seen in Table 3, cosmetic compositions 2 and 2 received higher scores than cosmetic composition 1. From there, it was confirmed that the peptides of Example 1 might be used for a cosmetic composition to reduce wrinkles, whiten skin, and rejuvenate skin.

TABLE 3

| Cosmetic composition | 1 | 2 | 3 |
|---|---|---|---|
| Skin irritation | X | X | X |
| Wrinkle reduction | 3.2 | 4.1 | 4.3 |
| Skin tone improvement | 3.1 | 4.3 | 4.4 |
| Skin rejuvenation | 2.9 | 3.9 | 4.3 |

Herein above, the applicants have described preferred examples of the present disclosure, but such examples are only examples that implement the technical idea of the present disclosure. Any changes or modifications should be construed as falling within the scope of the present disclosure as long as the changes or modifications embody the technical idea of the present disclosure.

1. A peptide comprising any one amino acid sequence represented by SEQ ID NO: 1 or 2.

2. The peptide of claim 1, wherein the peptide activates a retinoic acid receptor.

3. The peptide of claim 1, wherein the peptide is used to whiten skin, reduce a wrinkle, or rejuvenate skin.

4. A cosmetic composition comprising a peptide comprising any one amino acid sequence represented by SEQ ID NO: 1 or 2.

5. A composition to whiten skin, reduce a wrinkle, or rejuvenate skin, the composition comprising a peptide comprising any one amino acid sequence represented by SEQ ID NO: 1 or 2.

6. A cosmetic composition to whiten skin, the cosmetic composition comprising a peptide comprising any one amino acid sequence represented by SEQ ID NO: 1 or 2.

7. A cosmetic composition to reduce a skin wrinkle, the cosmetic composition comprising a peptide comprising any one amino acid sequence represented by SEQ ID NO: 1 or 2.


SEQUENCE LISTING (2 sequences)

SEQ ID NO: 1 - protein, 4 aa, synthetic construct
IQEM

SEQ ID NO: 2 - protein, 4 aa, synthetic construct
TVEF